(19) United States
(12) Patent Application Publication
(10) Pub. No.: US 2006/0069323 A1
(43) Pub. Date: Mar. 30, 2006

(54) Systems and methods for bi-lateral guidewire cannulation of branched body lumens

(75) Inventors: Jeffrey M. Elkins, Novato, CA (US); Harry B. Goodson IV, Fremont, CA (US); Aurelio Valencia, East Palo Alto, CA (US); Richard Aboytes, East Palo Alto, CA (US); Samir Patel, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FlowMedica, Inc., Fremont, CA (US)

(21) Appl. No.: 11/233,562

(22) Filed: Sep. 22, 2005

Related U.S. Application Data
(60) Provisional application No. 60/612,801, filed on Sep. 24, 2004.

(51) Int. Cl. A61M 25/00

(52) U.S. Cl. 600/585

(57) ABSTRACT

A system and method is provided that is adapted to allow for rapid cannulation of a guidewire into a branch lumen extending from a main lumen in a body of a patient, and in particular into two renal arteries extending from an abdominal aorta wall. A dual lumen catheter shaft delivers first and second pre-shaped guidewires to the location of the renal arteries in the aorta, such that the first and second pre-shaped guidewires self-cannulate within the renal arteries. Additional guidewires and/or interventional devices may be incorporated into the system and method for use with the catheter shaft, or over the two pre-shaped guidewires, to meet a particular need for a particular patient or intended procedure.

### CROSS-REFERENCES TO RELATED APPLICATIONS

This application claims the benefit of prior provisional patent application No. 60/612,801 (Attorney Docket No. 022352-002700US), filed on Sep. 24, 2004, the full disclosure of which is incorporated herein by reference.

This application is related to but does not claim priority from the following international applications which are incorporated herein by reference in their entirety: PCT/US01/13686 published as WO2001/83016A2; PCT/US03/21406; PCT/US03/29740 published as WO2004/026370A3; PCT/US04/08571; PCT/US03/29744 published as WO2004/032791A3; PCT/US03/29995 published as WO2004/030718A3; PCT/US03/29743 published as WO2004/026371A2; PCT/US03/29585 published as WO2004/034767A2; PCT/US03/29586; and PCT/US04/08573. This application is also related to but does not claim priority from the following U.S. applications which are incorporated herein by reference in their entirety: Ser. No. 09/229,390; Ser. No. 09/562,493; Ser. No. 09/724,691; and Ser. No. 10/251,915. This application is also related to U.S. Pat. No. 6,749,598 which is incorporated herein by reference.

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

This invention relates to the field of medical devices, and more particularly to a system and method for locally delivering materials within the body of a patient. Still more particularly, it relates to a system and method for locally delivering interventional medical devices into branch body lumens from a main lumen, and in particular delivering guidewires bilaterally into renal arteries or veins extending from an abdominal aorta or vena cava, respectively, in a patient.

2. Description of Related Art

Many challenges exist with conventional technology available to physicians who desire to perform renal artery diagnosis or intervention. In general, the conventional devices and methods require a relatively high level of skill, familiarity, and technique experience in ordered to cannulate even a single renal artery with a guidewire and catheter. This is because the renals typically have asymmetrical anatomical features and morphology, are located directly off of and somewhat perpendicular to the aorta, and are not easily accessed as the aorta is large relative to the renals. Thus, cannulation often uses backing and support off the opposite aortic wall to stabilize the catheter and guidewire tools to gain renal artery entrance. Additionally, there is significant variation among patients as to the exact locations, angles, and height differences among patients. Thus, a universal technique has been elusive to employ.

Current procedures to place such intravascular devices into the renal arteries or veins also involve the manipulation of guidewires and/or diagnostic or guiding catheters in the abdominal aorta/inferior vena cava in the area of the renal arteries/veins in order to gain access, and then following over (guidewires) or through (guiding catheters) these devices for placement of the intended interventional diagnostic, therapeutic, or prophylactic device. Such access procedures may require numerous expensive devices and be time consuming, increasing both the time of the procedure and its cost. As well, significant manipulation of various devices within the vasculature may lead to untoward clinical sequelae arising from trauma to the interior of the blood vessel walls or extensive x-ray or contrast media exposure.

Therefore, a need exists for a simpler, quicker, single device that may provide guidewire access to the renal vasculature for the delivery of interventional devices. There is in particular a need for such a device that may provide safe, quick, and easy access to both renals arteries or both renal veins simultaneously. Accordingly, there is also a need for an improved delivery device that is adapted to provide rapid, remote access for delivering interventional devices into a branch vessel extending at a unique location from a main vessel. There is in particular such a need for a bilateral delivery device assembly that is adapted to provide such access for interventional device delivery into multiple branch vessels extending at relatively unique locations from the main vessel. At least some of these needs will be met by the inventions described herein.

### BRIEF SUMMARY OF THE INVENTION

According to the present invention, methods and systems for positioning guidewires into branched lumens from a main vessel utilize a deployment catheter for manipulating the guidewires, either simultaneously or separately. In the methods of the present invention, the deployment catheter has a first lumen and a second lumen for receiving the first and second guidewires therein. The deployment catheter is positioned in the main vessel, such as an abdominal aorta, and the first guidewire is placed from the first guidewire lumen into a first targeted branched lumen and the second guidewire is placed from the second catheter lumen into a second targeted branched lumen. The targeted branch lumens are typically the right and left renal arteries, respectively. The deployment catheter may then be removed, typically in a proximal direction, from over the guidewires, leaving both guidewires available for over-the-wire placement of one or more catheters for diagnostic procedures, therapeutic procedures, or some combination thereof.

In a first specific embodiment of the methods of the present invention, the deployment catheter is axially advanced and/or retracted with the first and second guidewires extended laterally from a distal end thereof. The distal tips of the guidewires will be resilient or spring-like and oriented so that they simultaneously engage opposed regions of the main vessel wall. In this way, the guidewires apply generally equal, balanced forces against the main lumen wall and are able to enter the ostia of the branched target lumens when they reach the ostia.

Many times, axial movement of the deployment catheter will be sufficient in itself to place at least one and usually two of the guidewires into the branched ostia. In other cases, however, the branched ostia may not be axially aligned and/or rotationally aligned so that simultaneous movement of the lateral extensions of the guidewires do not automatically locate and enter the branched ostia. When that is the case, the individual guidewires can be manipulated relative to the deployment catheter, either while the deployment catheter is being moved or while it is stationary. In particular, the individual guidewires may be axially advanced and retracted relative to the deployment catheter in order to help position either or both of the guidewires into the target branched lumen. Alternatively or in addition, the guidewires may also be rotated about their own axes in order to help position the guidewire tips in the branched ostia.

In an alternate aspect of the methods of the present invention, the deployment catheter may be held stationary within the main vessel while the guidewires are individually advanced and manipulated, e.g., by rotating, in order to locate and enter the branched vessel through their respective ostia. The guidewires will typically viewed by fluoroscopic or other conventional techniques to assist in locating the branched luminal ostia. In all cases, after the guidewires have been positioned within the branched lumens, the deployment catheter may then be removed, leaving the guidewires available for subsequent catheter placement, as generally described above. The guidewires will usually each have deflected distal ends with a lateral extension, i.e., lateral distance from the axis of the guidewire when no forces are being applied, typically of at least 15 mm, preferably of at least 25 mm.

Preferably, the systems of the present invention will further comprise an introducer sheath. The introducer sheath may have a relatively short length, typically in the range from 5 cm to 25 cm, or may have a relatively long length, typically in the range from 20 cm to 60 cm, preferably from 30 cm to 45 cm. The use of long introducer sheaths can facilitate the introduction of the deployment catheter with the guidewires pre-advanced from a distal tip of the deployment catheter. In such cases, the laterally deflected distal ends of the guidewires will then be constrained within the long introducer sheath until they reach the general location of the target branched lumens, typically the renal arteries.

### BRIEF DESCRIPTION OF THE DRAWINGS

FIG. 1 illustrates a system constructed in accordance with the principles of the present invention including a dual lumen deployment catheter, a pair of guidewires having laterally deflected distal tips, and an optional introducer sheath.

FIG. 2 illustrates the dual lumen deployment catheter system of FIG. 1 having the pair of guidewires in place and further illustrates the ability to individually manipulate the guidewires with respect to the deployment catheter.

FIG. 3 illustrates the deployment catheter and guidewires, generally as shown in FIG. 2, used without an introducer sheath for placing the guidewires in the right and left renal arteries which branch from the abdominal aorta.

FIGS. 4A to 4D illustrate the removal of the deployment catheter from a deployed pair of guidewires in the renal arteries to expose the guidewires and utilize the guidewires for delivering a therapeutic or interventional catheter to one of the renal arteries.

FIGS. 5A and 5B illustrate use of a long sheath for deploying guidewires according to the methods of the present invention.

FIGS. 6A to 6D illustrate use of a short sheath for deploying catheters in accordance with the methods of the present invention.

FIG. 7 illustrates deployment of guidewires into renal arteries which are generally aligned, while FIG. 8 illustrates deployment of the guidewires into renal arteries which are not axially aligned.

FIGS. 9 and 10 illustrate the advantages of being able to rotate the individual guidewires relative to the deployment catheter to access renal arteries which are rotationally displaced in an anterior-posterior plane.

### DETAILED DESCRIPTION OF THE INVENTION

According to one present embodiment, a catheter/guidewire based system is provided that is adapted to gain rapid guidewire access to the renal arteries, such as for example for the purposes of renal diagnostic angiograms and renal intervention (e.g., percutaneous transluminal angioplasty or “PTA”, stent placement, etc.). These wires are then in place to allow catheters and other catheter type tools to be advanced over them, such as for example after a dual lumen deployment catheter is removed from the blood vessels or other body lumens, as will be explained in further detail below.

In a further detailed embodiment, systems of the present invention include the deployment catheter and a pair of pre-shaped guidewires (for example typically between about 0.014″ and 0.038″ in diameter). These guidewires are held in general spatial relationship together via the dual lumen deployment catheter. The dual lumen deployment catheter is used to keep the two individual shaped wires in a generally straightened configuration to facilitate introduction and manipulation in the target body lumens as discussed below. Thus, the system allows for rapid bilateral cannulation of renal arteries or other branched target lumens, but can also be used for very rapid single renal artery cannulation when desired, such as for example utilizing only one directional aspect of a dual wire delivery system, or in another example using a second dummy arm as elsewhere disclosed herein for biased delivery catheter branch arm delivery. Or, one lateral delivery aspect may incorporate guidewire cannulation, whereas the second lateral delivery aspect may incorporate delivery lumen catheter cannulation. Once cannulation is achieved, the dual lumen catheter is drawn proximally of the wires (removed), leaving the wires in place. Then, the physician can advance whatever tool is desired over the now cannulated guidewire.

The dual wire and deployment catheter systems of the present invention provide substantial benefits over conventional technologies and methods. In one regard, the dual wires respectively provide a “built in” supportive backing against the opposing aortic wall or renal ostium. While the bifurcated delivery catheter systems of the prior applications which have been referred above can directly place shaped catheters, the present invention places guidewires instead of delivery catheter arms, thus allowing for other catheter tools to be used in conjunction with these wires, as they can be advanced over these wires as needed.

The deployment catheter holds the guidewires in a proper position (e.g., approximately 180 degree opposed alignment) for placement. By advancing the deployment catheter toward the distal end of the wires, the wires and catheter can behave as a single unit when desired, but also allow movement and alignment of individual wires as needed. Such adjustability includes for example up or down movement, and torque independently or together via rotation of the dual lumen holding catheter. This adjustability is well adapted for use in difficult anatomy where independent movement of wires may be necessary.

The systems of the present invention incorporating two shaped wires and the dual lumen deployment catheter can be advanced through either a standard, commercially-available sheath or custom designed delivery sheath, such as elsewhere herein described, for bilateral guidewire delivery to the renals. Or, the catheter shaft can be advanced over a single guidewire, including one of the system's own wires, or over a commercially available wire.

Once in position, the guidewires can be adjusted to a “self guiding” configuration, wherein they are adapted to cannulate the respectively spaced renal ostia by seeking to be spread open and navigate into the chamfered/radiused entrances with minimal torque and advancement. Such may be accomplished for example by self-expanding or spring-like recovery from respectively constrained configurations within the dual lumens of the delivery catheter, to respectively unconstrained memory configurations having shapes that are respectively biased away from each other toward the renal ostia along the aortic wall. In addition or alternative to the self-guiding mode, the wires may also be individually manipulated, which may be necessary for severely difficult anatomy or in the case of stenotic lesions.

The wires of the present embodiments may be constructed of typical guidewire materials, including for example stainless steel, or a superelastic or shape memory alloy such as nickel-titanium alloy, e.g. Nitinol. The wires may also be coated with a lubricious coating, such as for example polytetrafluoroethylene (PTFE), a hydrophilic coating, or another suitable lubricous coating. Furthermore, in highly beneficial illustrative embodiments the wires are pre-shaped, and in particular beneficial embodiments are shaped to have the combined appearance similar to a “Y” when placed together.

The dual lumen deployment catheter of the present invention is made of various conventional catheter shaft materials, such as for example of a polymer typical of catheters. In addition, the catheter can also employ a lubricous coating within the respective guidewire lumens, to allow easy removal and/or advancement of wires. In the present embodiments, the dual-lumen catheter is not adapted for cannulation into either renal artery, but rather another catheter would be incorporated into the overall system after removal (e.g. retraction over the wire) of the system's dual lumen catheter.

It is to be appreciated that various modifications may be made to the present illustrative embodiments for rapid guidewire cannulation without departing from various aspects herein contemplated for the invention. For example, various materials, coatings, dimensions, lengths, and respective configurations and spatial arrangements may be incorporated into either or both the guidewires, catheter shaft, or components thereof of the embodiments which differ than those specifically herein described. Moreover, the number of guidewires, and lumens of the catheter shaft accordingly, may be modified to suit a particular need. For example, as previously described above, a single guidewire may be used in the various embodiments for single ostium cannulation. Moreover, a design providing for three or more wires and/or respective catheter lumens may be employed for special cases where more than two ostia are to be cannulated. In addition, the systems and methods may be adapted for use in other anatomies and for other indications than for renal cannulation.

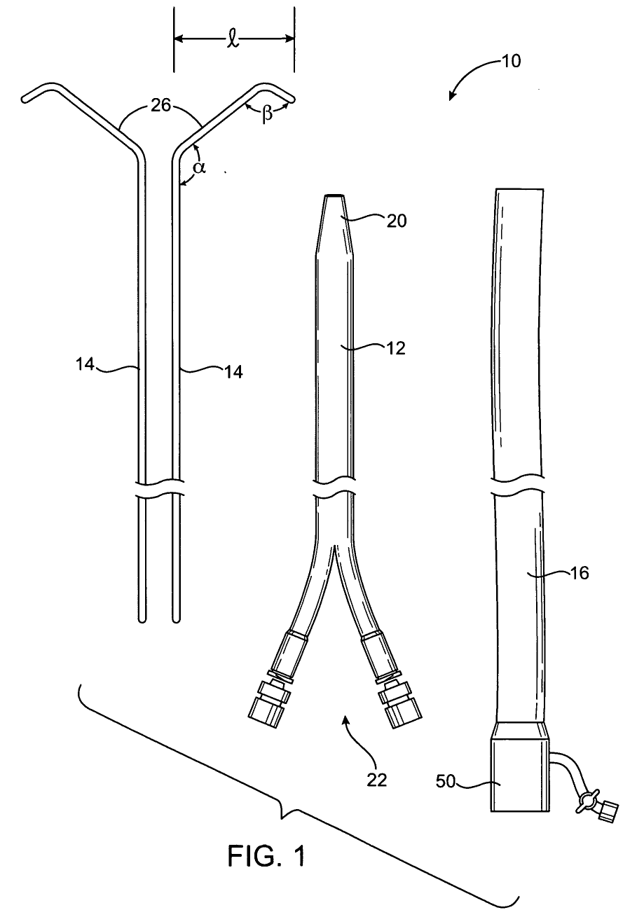

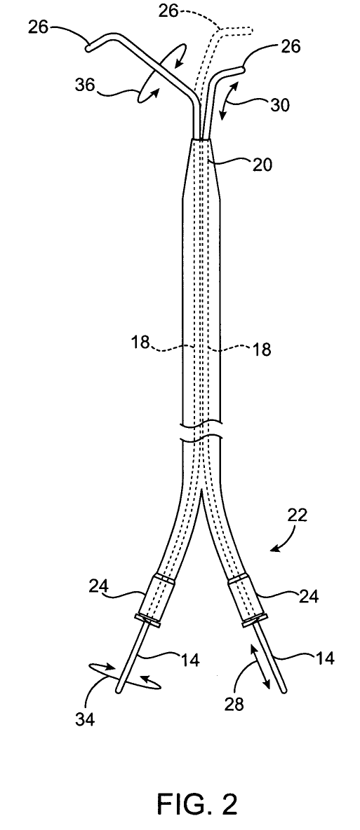

Referring now to FIG. 1, a guidewire deployment system 10 according to the present invention includes a dual lumen deployment catheter 12, a pair of guidewires 14 and optionally an introducer sheath 16. The deployment catheter 12 includes a pair of internal lumens 18 which removably receive the individual guidewires 14, as best seen in FIG. 2. The deployment catheter 12, in turn, may be introduced to a patient's vasculature or other luminal structure through an internal lumen of the introducer sheath 16, as will described in more detail below.

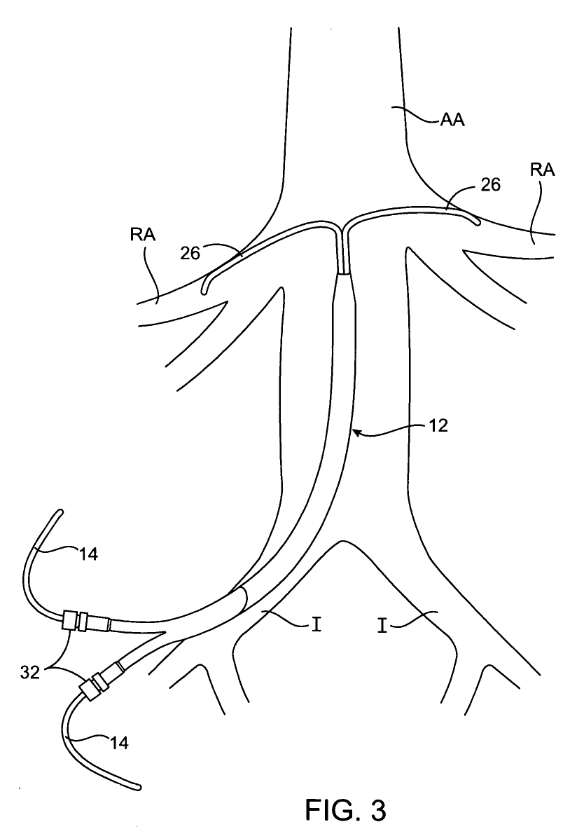

The deployment catheter 12 may be constructed in a variety of ways. For example, it may be formed as a single dual lumen extrusion typically having a tapered distal end 20 and a bifurcated proximal end 22. Alternatively, the deployment catheter 12 could be formed from a pair of single lumen extrusions which are attached or otherwise held together along their proximal lengths, for example by a coaxial outer cover or sheath. In all cases, the internal lumens 18 will typically terminate at their proximal ends in a hemostatic or other valve structure 24 which permits selective introduction and manipulation of the individual guidewires 14 through the catheter so that shaped distal ends 26 of each guidewire may be advanced from the distal end 20 of the catheter and individually manipulated, as shown in FIG. 2. For example, each guidewire 14 may be axially advanced and retracted by manipulating a proximal end of the guidewire 14, optionally using removable positioning clamps 32, as shown in FIG. 3 (where valves 24 are not shown). In this way, axial movement of the proximal end of the guidewire 14, as shown by arrow 28, results in a corresponding axial movement of the distal end of the guidewire, as shown by arrow 30. Similarly, rotational movement of the proximal end of the guidewire 14, as shown by arrow 34 results in a corresponding rotational movement of the shaped distal end 26 of the guidewire, as shown by arrow 36. Using these manipulations, as well as axial advancement of the deployment catheter 12 itself, the shaped distal ends 26 of each guidewire 14 may be advanced through an access site in an iliac artery I, through the lower abdominal aorta, and into the renal arteries RA, as shown in FIG. 3. Details of specific protocols for such advancement are discussed below.

The guidewires 14 may be formed from conventional guidewire materials, as described generally above. These specific geometry and dimensions of the shaped distal ends 26 will be chosen based on the bifurcated body lumens which are being targeted. In the case of the renal arteries, a preferred geometry is shown in FIG. 1, where the shaped distal end has a first bend with an angle a in the range from 90° to 140° and a second bend with an angle β in the range from 80° to 120°. Total lateral extension of the shaped distal end from the axis of the guidewire body to the tip of the guidewire typically has a length l in the range from 15 mm to 50 mm, preferably from 25 mm to 40 mm.

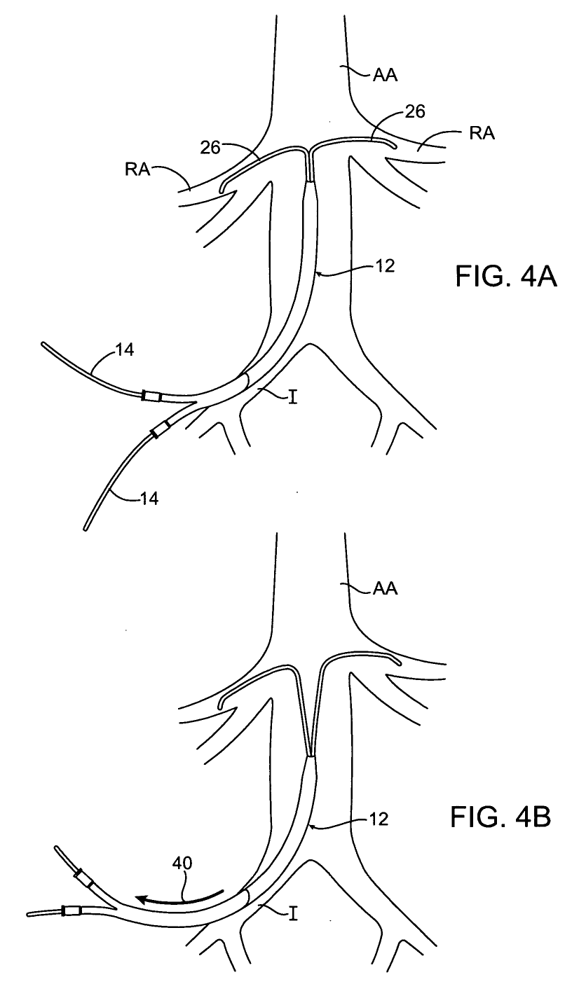

Referring now to FIGS. 4A through 4D, the deployment catheter 12 will typically be removed from the guidewires 14 after the shaped distal ends 26 are in place in the renal arteries RA. Initially, the deployment catheter 12 will contain the proximal portions of the guidewires 14, as shown in FIG. 4A. The deployment catheter 12 will then be withdrawn proximally in the direction of arrow 40, as shown in FIG. 4B. Typically, an introducer will be in place to provide access into the iliac artery I, but the introducer is not shown in FIGS. 4A through FIGS. 4D for simplicity. After the deployment catheter 12 has been removed, the guidewires 14 remain in place providing access from the iliac artery I to the renal arteries RA, as shown in FIG. 4C. Again, usually an introducer will be in place to establish access into the iliac artery. Using the exposed, available guidewires, various catheters and catheter-like devices may be introduced over the guidewires 14 and placed in the renal arteries RA, as shown by exemplary catheter C in FIG. 4D.

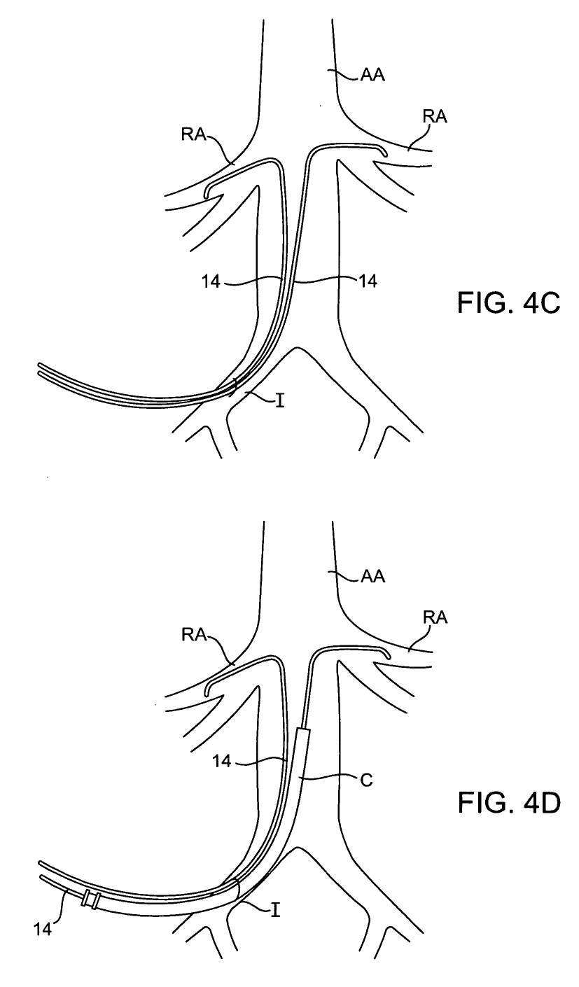

Referring now to FIGS. 5A and 5B, in some instances, it will be desirable to introduce the deployment catheter 12 through a relatively long introducer sheath 16′. Using such a long introducer, typically having a length in the range from 30 cm to 45 cm for guidewires being introduced from an iliac artery I to the renal arteries RA, the guidewires 14 may be advanced from the distal end 20 of the deployment catheter 12 prior to being released into the abdominal aorta AA, as shown in FIG. 5B. In such instances, when the shaped distal ends 26 of the guidewires 14 emerge from the distal end 52 of the introducer sheath 16′, they will immediately deploy outwardly as a result of their own spring force. The ends 26 may then be advanced into the renal arteries RA either by axial advancement and/or rotation of the deployment catheter 12, or by axial advancement and/or rotation of each individual guidewire relative to the deployment catheter, or by some combination thereof. As the physician will typically be able to observe the position of the guidewires under fluoroscopy, the system of the present invention provides many opportunities to position and reposition the guidewires 14, either simultaneously or individually.

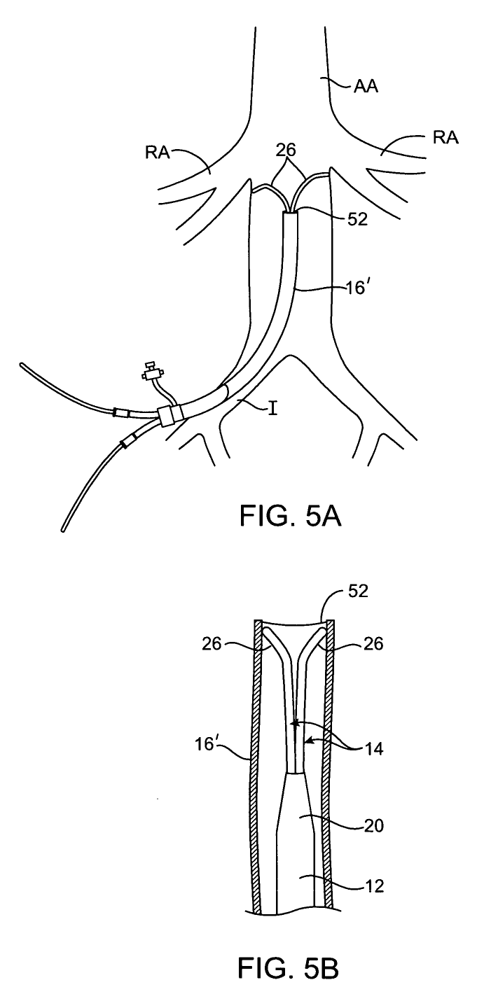

Referring now to FIGS. 6A to 6D, use of a short introducer sheath 16″ for introducing the deployment catheter 12 and guidewires 14 will be described. Initially, a guidewire is placed through the short introducer sheath 16″ and advanced to the region of the renal arteries RA, as shown in FIG. 6A. The guidewire may be a conventional guidewire or optionally may be one of the guidewires 14 which are part of the system 10 of the present invention. Once the deployment catheter 12 is in place, as shown in FIG. 6B, the guidewires 14 will be extended from the distal end 20. The conventional guidewire GW had been used for placement, that guidewire may be exchanged for a guidewire 14, and a second guidewire 14 introduced through the other lumen. The shaped distal ends 16 of the guidewires 14 may then be further advanced, as shown in FIG. 6C, and manipulated individually, simultaneously, and/or in combination with manipulation of the deployment catheter 12 in order to position the shaped ends 26 into the renal arteries RA, as shown in FIG. 6D.

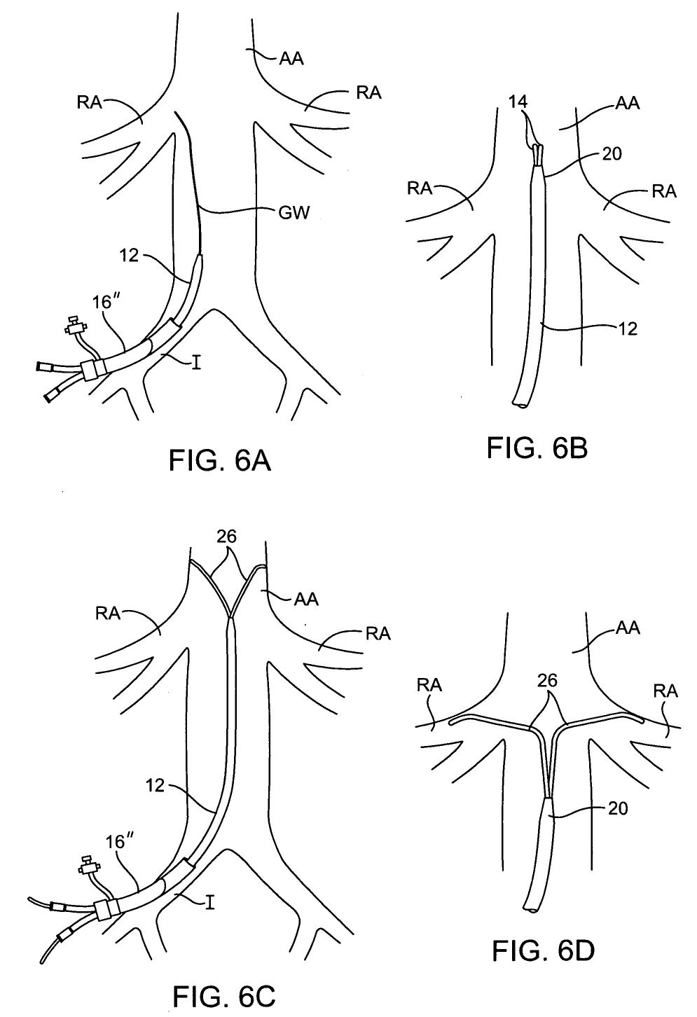

Referring now to FIGS. 7 and 8, the positioning of the shaped distal ends 26 of the guidewires 14 in different patient anatomies can be described. In a “normal” anatomy, the renal arteries RA will typically be nearly directly opposed on opposite sides of the abdominal aorta AA, as shown in FIG. 7. In those instances, placement of the guidewire shaped ends 26 will be relatively straightforward. In other instances, the renal arteries RA may be significantly axially displaced, as shown in FIG. 8. In those instances, the ability to individually manipulate the distal ends 26 of the guidewires 14 will be a substantial advantage. In particular, a first of the shaped ends 26 may be introduced into a first of the renal arteries RA and left in place while the deployment catheter 12 is repositioned, allowing a second of the shaped distal ends 26 to be introduced into the second of the renal arteries RA.

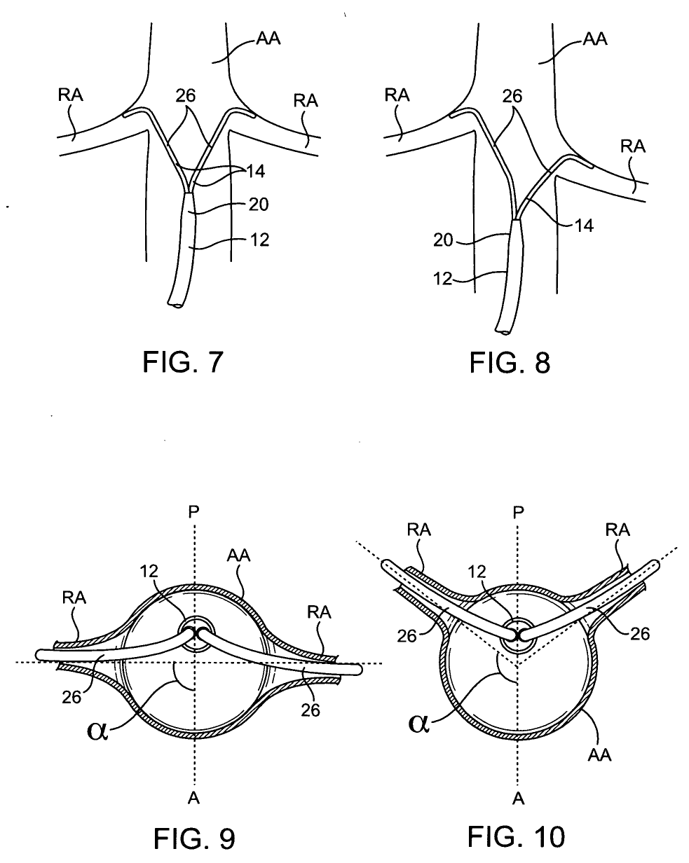

Referring now to FIGS. 9 and 10, in addition to axial displacement of the renal arteries RA, the renal arteries RA may also be displaced rotationally relative to the anterior-posterior plane AP. As shown in FIG. 9, the renal arteries RA may be generally opposed to each other at a generally right angle α relative to the anterior-posterior plane AP. In other instances, however, as shown in FIG. 10 the renal arteries RA may be at an angle α which is much greater than 90°. The ability to independently rotate the guidewires 14 and orient the shaped distal ends 26 greatly facilitates access to such rotationally offset renal arteries.

Additional modifications or improvements may be made by the embodiments shown and described herein without departing from the intended scope of the invention which is considered to be broadly beneficial according to various independent aspects described. For example, various modifications to or combinations with the present embodiments may be made in view of other available information to one of ordinary skill in the art upon review of this disclosure and remain within the intended scope of the invention.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A method for positioning guidewires into branched lumens from a main vessel, said method comprising:
providing a deployment catheter having first and second lumens and first and second guidewires positioned in said first and second lumens, respectively;
positioning the deployment catheter in the main vessel;
placing the first guidewire in a first target branched lumen and the second guidewire in a second target branched lumen; and
removing the deployment catheter from the guidewires leaving both guidewires available for over-the-wire placement of a catheter.

2. A method as in claim 1, wherein placing the guidewires comprises axially advancing and/or retracting the deployment catheter within the main vessel with the first and second guidewires extended laterally from a distal end of the deployment catheter so that at least one of the guidewires enters the target branched lumen by its spring force.

3. A method as in claim 2, wherein placing the guidewires further comprises axially advancing the guidewires relative to the deployment catheter to help position either or both guidewires in the target branched lumen.

4. A method as in claim 3, wherein placing the guidewires further comprises rotating at least one of the guidewires about its axis to help position either or both guidewires in the target branched lumen.

5. A method as in claim 1, wherein placing the guidewires comprises axially advancing the guidewires relative to the deployment catheter after the deployment catheter has been positioned in the main vessel.

6. A method as in claim 5, wherein each guidewire is advanced separately.

7. A method as in claim 5, further comprising rotating at least one guidewire about its axis to help position the guidewire in the target branched vessel.

8. A method as in claim 7, further comprising axially advancing and/or retracting the deployment catheter within the main vessel to assist in positioning the guidewire(s) in the target branched lumen(s).

9. A method as in claim 1, wherein the main lumen and branched lumens are blood vessels.

10. A method as in claim 9, wherein the main lumen is an abdominal aorta and the branched lumens are the right and left renal arteries.

11. A method as in claim 1, further comprising advancing at least one catheter over at least one of the placed guidewires and into one of the target branched lumens.

12. A method as in claim 11, further comprising advancing at least a second catheter over the other of the placed guidewires and into the other of the branched lumens.

13. A system for deploying catheters from a main lumen into branched lumens, such system comprising:
a deployment catheter having a proximal end, a distal end, and at least a first lumen and a second lumen therethrough;
a first guidewire having a length greater than that of the deployment catheter; and
a second guidewire having a length greater than that of the deployment catheter.

14. A system as in claim 13, wherein the deployment catheter has a first hemostatic valve at a proximal end of the first lumen and a second hemostatic valve on a proximal end of the second lumen.

15. A system as in claim 14, wherein the deployment catheter has a length in the range from 30 cm to 45 cm, a width in the range from 1 mm to 4 mm, and a diameter for each lumen in the range from 0.3 mm to 1.3 mm.

16. A system as in claim 15, wherein the first and second guidewires each have deflected distal ends with a lateral extension of at least 25 mm.

17. A system as in claim 1, further comprising an introducer sheath.

18. A system as in claim 17, wherein the introducer sheath is short having a length in the range from 10 cm to 25 cm.

19. A system as in claim 17, wherein the introducer sheath is long having a length in the range from 20 cm to 50 cm.
